(19) United States
(12) Patent Application Publication
(10) Pub. No.: US 2004/0171650 A1
(43) Pub. Date: Sep. 2, 2004

(54) Novel anthelmintic and insecticidal compositions

(76) Inventors: Byung Hyun Lee, Kalamazoo, MI (US); Martha Jane Larsen, Kalamazoo, MI (US); Teresa Maria Kubiak, Richland, MI (US)

Correspondence Address: PHARMACIA & UPJOHN, 301 HENRIETTA ST, 0228-32-LAW, KALAMAZOO, MI, 49007, US

(21) Appl. No.: 10/776,837

(22) Filed: Feb. 11, 2004

Related U.S. Application Data
(60) Provisional application No. 60/449,239, filed on Feb. 21, 2003.

(51) Int. Cl. A61K 31/4439; A61K 31/4196; C 07D 4 3/02

(52) U.S. Cl. 514/341; 514/384; 546/272.4; 548/263.2

(57) ABSTRACT

The present invention relates to novel anthelmintic and insecticidal compositions in general, and, more specifically, compositions containing triazole derivatives as active ingredients.

### CROSS REFERENCE TO RELATED APPLICATION

This application claims the benefit of U.S. provisional application Serial No. 60/449,239 filed on 21 Feb. 2003, under 35 USC 119(e)(i), which are incorporated herein by reference in their entirety.

### BACKGROUND OF THE INVENTION

1. Field of the Invention

The present invention relates to novel anthelmintic and insecticidal compositions in general, and, more specifically, compositions containing triazole derivatives as active ingredients.

2. Technology Description

Control of parasitic infections in human and animal populations remains an important global endeavor. The causative organisms may be categorized as endoparasitic members of the classes Nematoda, Cestoidea and Trematoda or phylum Protozoa, or as ectoparasitic members of the phylum Arthropoda. The former comprises infections of the stomach, intestinal tracts, lymphatic system, tissues, liver, lungs, heart and brain. Examples include trichinosis, lymphatic filariasis, onchocerciasis, schistosomiasis, leishmaniasis, trypanosomiasis, giardiasis, coccidiosis and malaria. The latter ectoparasites include lice, ticks, mites, biting flies, fleas and mosquitoes. These often serve as vectors and intermediate hosts to endoparasites for transmission to human or animal hosts. While certain helminthiases can be treated with known drugs, evolutionary development of resistance necessitates a further search for improved efficacy in next generation anthelmintic agents.

The control of ectoparasites, such as fleas, ticks, biting flies and the like, has long been recognized as an important aspect of human and animal health regimens. Traditional treatments were topically applied, such as the famous dips for cattle, and indeed such treatments are still in wide use. The more modern thrust of research, however, has been towards compounds, which can be administered orally, or parenterally to the animals and which will control ectoparasitic populations by poisoning individual parasites when they ingest the blood of a treated animal.

The control of endoparasites, especially intestinal parasites, has also been an important aspect of human and animal health regimens. Although a number of ectoparasiticides and endoparasiticides are in use, these suffer from a variety of problems, including a limited spectrum of activity, the need for repeated treatment and, in many instances, resistance by parasites. The development of novel endo- and ectoparasiticides is therefore essential to ensure safe and effective treatment of a wide range of parasites over a long period of time.

Despite the above teachings, there still exists a need in the art for treatment of pests.

The allatostatins are an important group of insect neurohormones controlling diverse functions including feeding, locomotion, nutrient absorption, reproduction, growth and sensory perception (Nichols, R., J.Neurogenetics, 2002, 16, 1-28; Birgulet al., The EMBO J., 1999, 18, 5892-5900; Lenz et al., Biochem. Biophys. Res. Comm. 2000, 273, 1126-1131).

### BRIEF SUMMARY OF THE INVENTION

In accordance with the present invention, a novel composition of matter which is capable of treatment of pests is provided. The composition contains triazole derivatives of Formula I:

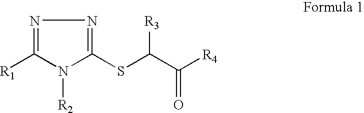

wherein R₁, R₂ and R₄ are independently selected from the group H, C₁-C₈ alkyl, phenyl, substituted phenyl, benzyl, substituted benzyl, heteroaryl, substituted heteroaryl, hetroarylmethylene, and substituted hetroarylmethylene;

R₃ is H, C₁-C₈ alkyl, heteroalkyl, cycloalkyl, aryl, heteroaryl; as active ingredients. Further, the invention provides compositions containing a compound of Formula I and the use of compounds of Formula I and their compositions as insecticides and anthelmintics.

### DETAILED DESCRIPTION OF THE INVENTION

Definitions

In the description, the following terms are used. The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₈ means 1-8 eight carbons. Examples of saturated hydrocarbon radicals include groups such as methyl, ethyl, n-propyl, isopropyl, n-butyl, t-butyl, isobutyl, sec-butyl, cyclohexyl, (cyclohexyl)ethyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, I- and 3-propynyl, 3-butynyl, and the higher homologs and isomers. The term “alkylene” by itself or as part of another substituent means a divalent radical derived from an alkane, as exemplified by —CH2CH2CH2CH2—.

The terms “alkoxy . . . alkylcylamino” and “alkylthio” refer to those groups having an alkyl group attached to the remainder of the molecule through an oxygen, nitrogen or sulfur atom, respectively. Similarly, the term “dialkylamino” is used in a conventional sense to refer to —NRR wherein the R groups can be the same or different alkyl groups.

The term “heteroalkyl,” by itself or in combination with another term, means, unless otherwise stated, a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Examples include —CH2—CH2—O—CH3, —CH2—CH2—NH—CH3, —CH2—CH2—N(CH3)—CH3, —CH2—S—CH2—CH3, —CH2—CH2—S(O)—CH3, —CH2—CH2—S(O)2—CH3, —CH═CH—O—CH3, —Si(CH3)3, —CH2—CH═N—OCH3, and —CH═CH—N(CH3)—CH3. Up to two heteroatoms may be consecutive, such as, for example, —CH2—NH—OCH3. Also included in the term “heteroalkyl” are those radicals described in more detail below as “heterocycloalkyl.” The term “heteroalkylene” by itself or as part of another substituent means a divalent radical derived from heteroalkyl, as exemplified by —CH2—CH2—S—CH2CH2— and —CH2—S—CH2CH2—NH—CH2—. For heteroalkylene groups, heteroatoms can also occupy either or both of the chain termini. Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl. include cyclopentyl, cyclohexyl, 1-cyclohexenyl, 3-cyclohexenyl, cycloheptyl, and the like. Examples of heterocycloalkyl include 1-piperidinyl, 2-piperidinyl, 3-piperidinyl, 4-morpholinyl, 3morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “Fluoroalkyl,” are meant to include monofluoroalkyl and polyfluoroalkyl.

The term “aryl,” employed alone or in combination with other terms (e.g., aryloxy, arylthioxy, aralkyl) means, unless otherwise stated, an aromatic substituent which can be a single ring or multiple rings (up to three rings) which are fused together or linked covalently. The term “heteroaryl” is meant to include those aryl rings which contain from zero to four heteroatoms selected from N, O, and S, wherein the nitrogen and sulfur atoms are optionally oxidized, and the nitrogen atom(s) are optionally quaternized. The “heteroaryl” groups can be attached to the remainder of the molecule through a heteroatom. Non-limiting examples of aryl and heteroaryl groups include phenyl, 1-naphthyl, 2-naplithyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5- isoquinolyl, 2-quinoxalinyl, 5-quinoxalinyl, 3-quinolyl, and 6-quinolyl.

Substituents for each of the above noted aryl ring systems are selected from the group of acceptable substituents described below. The term “aralkyl” is meant to include those radicals in which an aryl or heteroaryl group is attached to an alkyl group (e.g., benzyl, phenethyl, pyridylmethyl and the like) or a heteroalkyl group (e.g., phenoxymethyl, 2-pyridyloxymethyl, 3-(1-naphthyloxy)propyl, and the like). Each of the above terms (e.g., “alkyl”, “cycloalkyl”, “heteroalkyl”, “heteroaryl” “aryl” “alkoxy”, “alkylamino”, “alkylcycloamino” “dialkylamino” and “alkylthio”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, —halogen, —SiR′R″R, —OC(O)R′, —C(O)R′, —CO₂R′, CONR′R″, —OC(O)NR′R″, —NR′C(O)R′, —NR′—C(O)NR″R′″, —NR′COOR′, —NH—C(NH2)═NH, —NR′C(NH2)═N—H, —NH—C(NH2)═NR′, —S(O)R′, S(O)₂R′, —S(O)₂NR′R″, —CN and —NO₂ in a number ranging from zero to (2N+1), where N is the total number of carbon atoms in such radical. R′, R″ and X″ each independently refer to hydrogen, unsubstituted (Cl—COalkyl and heteroalkyl, unsubstituted aryl, aryl substituted with 1-3 halogens, unsubstituted alkyl, alkoxy or thioalkoxy groups, or aryl-(C1-C4)alkyl groups. When R′ and R″ are attached to the same nitrogen atom, they can be combined with the nitrogen atom to form a 5-, 6-, 7 or 7-membered ring. For example, —NR′R″ is meant to include 1-pyrrolidinyl and 4morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Similarly, substituents for the aryl groups are varied and are selected from: halogen, —OR′, —OC(O)R′, —NR′R″, —SR′, —R′, —CN, —NO2, —CO2R′, —CONR′R:′, —C(O)R′, —OC(O)NR′R:′, —NR″C(O)R′, —NR″C(O)₂R′, —NR′—C(O)NR″R′″, —NH—C(NH2)═NH, —NR′C(NH2)═NH, —NH—C(NH2)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl, (unsubstituted aryl)-(C₁-C₄)alkyl, and (unsubstituted aryloxy-(C1-C4)alkyl.

Two of the substituents on adjacent atoms of the aryl ring may optionally be replaced with a substituent of the formula —T—C(O)—(CH₂)q—U—, wherein T and U are independently —NH—, —O—, —CH₂— or a single bond, and the subscript q is an integer of from 0 to 2. Alternatively, two of the substituents on adjacent atoms of the aryl ring may optionally be replaced with a substituent of the formula —A—(CH₂),—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl ring may optionally be replaced with a substituent of the formula —(CH2),—X—(CH₂)t—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

As used herein, the term “heteroatom” is meant to include oxygen (O), nitrogen (N), ) and sulfur(S).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent.

Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactouronic acids and the like (see, for example, Berge et al. (1977) J. Miami; Sci. 66:1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner.

The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. The term “prodrug” denotes a derivative of a known direct acting drug, which derivative has enhanced delivery characteristics and therapeutic value as compared to the drug, and is transformed into the active drug by an enzymatic, for example by hydrolysis in blood, or chemical process [see T. Higuchi and V. Stella, “Pro-drugs as Novel Delivery Systems,” Vol. 14 of the A.C.S. Symposium Series; Bioreversible Carriers in Drug Design, ed. Edward B. Roche, American Pharmaceutical Association and Pergamon Press, (1987); Notari, R. E., “Theory and Practice of Prodrug Kinetics,” Methods in Enzymology, 112:309-323 (1985); Bodor, N., “Novel Approaches in Prodrug Design,” Drugs of the Future, 6(3): 165-182 (1981); and Bundgaard, H., “Design of Prodrugs: Bioreversible-Derivatives for Various Functional Groups and Chemical Entities,” in Design of Prodrugs (H.

Bundgaard, ed.), Elsevier, N.Y. (1985)]. The prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). As used herein, a “prodrug” is any covalently bonded carrier that releases in vivo the active parent drug according to the Formula I when such prodrug is administered to the subject. Prodrugs of the compounds of Formula I are prepared by modifying functional groups present on the compound in such a way that the modifications are cleaved, either in routine manipulation or in vivo, to the parent compound. Prodrugs include, but are not limited to, compounds derived from compounds of Formula I wherein hydroxy, amine or sulfhydryl groups are bonded to any group that, when administered to the subject, cleaves to form the free hydroxyl, amino or sulfhydryl group, respectively. Selected examples include, but are not limited to, biohydrolyzable amides and biohydrolyzable esters and biohydrolyzable carbamates, carbonates, acetate, formate and benzoate derivatives of alcohol and amine functional groups. Furthermore, prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of Formula I and Formula II. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (3 H), iodine-125 (125j) or carbon-14 (14C). All isotopic variations of the compounds of the present invention, whether radioactive or not, are intended to be encompassed within the scope of the present invention.

Detailed Description of the Preferred Embodiment

The present invention provides a compound of Formula I comprising:

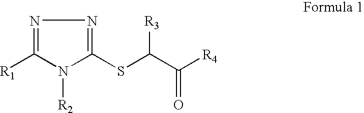

wherein R₁, R₂ and R₄ are independently selected from the group comprising H, C₁-C₈ alkyl, phenyl, substituted phenyl, benzyl, substituted benzyl, heteroaryl, substituted heteroaryl, hetroarylmethylene, and substituted hetroarylmethylene;

R₃ is H, C₁-C₈ alkyl, heteroalkyl, cycloalkyl, aryl, heteroaryl;

compositions containing a compound of Formula I and the use of compounds of Formula I and their compositions as insecticides and anthelmintics.

The general synthetic sequence depicted in Scheme I is useful in making compounds of the present invention.

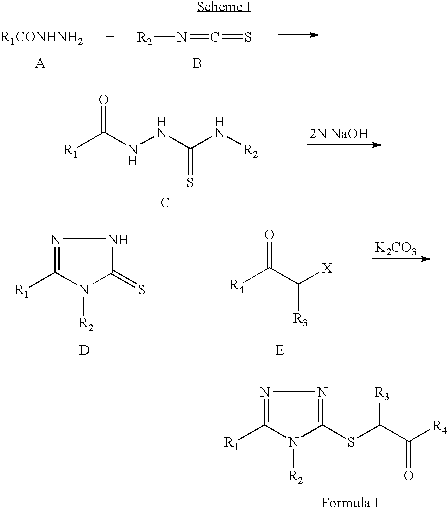

In Scheme I, R₁, R₂, R₃ and R₄ are as described for Formula I;

X is selected from the group —Cl, —Br, —I, —OSO₂R₅

R₅ is methyl, phenyl, tolyl or trifluromethyl.

Contacting an isothiocyanate of Formula B with an acid hydrazide of Formula A provides a thiosemicarbazide of Formula C which is cyclized with base to give the triazolethione of Formula D as has been described (see for example: Jaiswal, R. K., et.al., J.Heterocycl. Chem., 1979, 16, 561; Lee, U.S. Pat. No. 5,498,720; Connor et.al., U.S. Pat. No. 5,489,598; Gall et.al., U.S. Pat. No. 4,481,360). Reaction of D with a ketone of Formula E gives the compounds of the invention, Formula I as has been described (see for example: Babichev, F. S., et.al., Khimiya Geterotsiklicheskikh Soedinenii 1977, 8, 1132; Lee, op.cit.; Knish, E. G.; et.al., Farmatsevtichnii Zhurnal 1983, 2, 64; Gulerman, N. N., et.al., Farmaco (2001), 56(12), 953-958).

Compounds of the invention were evaluated in an allatostatin receptor binding assay as has been described (Larsen, M. J. et. al., Biochemical and Biophysical Reseach Communications, 2001, 286, 895-901; Lowery, D. E., et. al., 2001, WO 01/31005). Anthelmintic activity was evaluated in a Ascaris suum muscle tension assay as has been described (Bowman, J. W., et. al., J Neurophys., 1995, 74(5), 1880-8; Davis, R. E., et. al., J. Neurosci., 1989, 9, 403-414).

### EXAMPLES

Without further elaboration, it is believed that one skilled in the art can, using the preceding descriptions, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize variations from the procedures both as to reactants and as to reaction conditions and techniques.

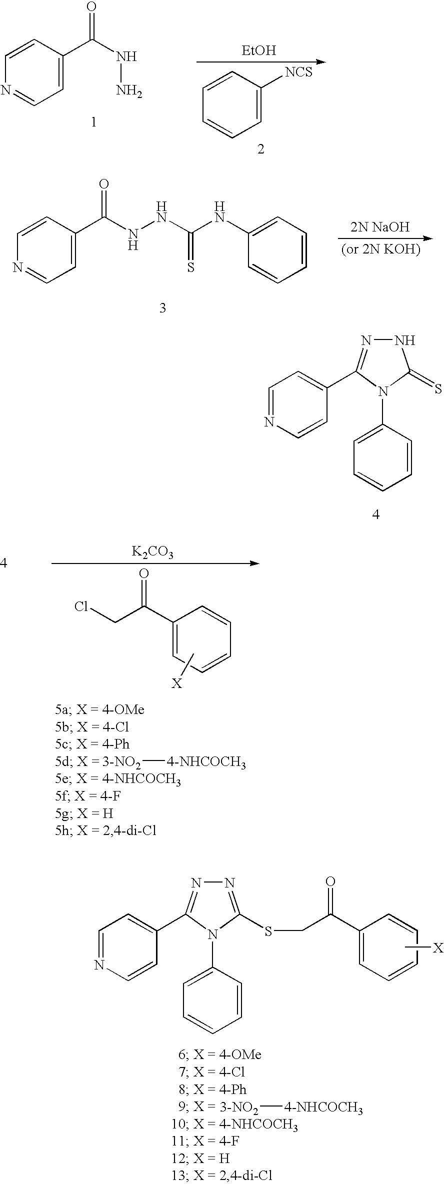

### Preparation of 3 from 1

Isonicotinic hydrazide (1, 4.58 g, 33.4 mmol) and phenyl isothiocyanate (2, 4.52 g, 33.4 mmol) are refluxed in ethanol for 2 h. After the mixture is cooled down to room temperature (rt), the white precipitate (8.92 g) is collected by filtration. Physical characteristics: MS (ES+) for m/z 272 (M+H).

### Preparation of 4 from 3

Compound 3 (8.19 g, 30 mmol) is refluxed in 2 N NaOH (aqueous solution, 60 mL) for 5 h. After the reaction mixture is cooled down to rt, 3 N hydrochloric acid (70 mL) is added to acidify the solution. The white precipitate is collected by filtration, washed with distilled water and dried (6.62 g).

Physical characteristics: MS (ES−) for m/z 253 (M−H).

### Example 1

### Preparation of 6

Compound 4 (35 mg, 0.14 mmol), 2-CHloro-1-(4′-methoxyphenyl)-ethanone (5a, 25 mg, 0.14 mmol), and potassium carbonate (19 mg, 0.14 mmol) are refluxed in acetone (15 mL) for 3 h. After cooling down to rt, the reaction mixture is poured into brine (40 mL). The mixture is then extracted with methylene chloride (40 mL). The organic layer is dried (Na₂SO₄) and concentrated. The residue is chromatographed on a silica plate by elution with 5% methanol in methylene chloride. The desired compound is isolated as a white solid (54 mg, 97% yield).

Physical characteristics: MS (ES+) for m/z 403 (M+H)⁺; ¹H NMR (CDCl₃) δ 8.54, 8.03, 7.56, 7.31, 6.96, 4.97, 3.88.

### Example 2

### Preparation of 7

Compound 4 (42 mg, 0.165 mmol), 4-chlorophenacyl chloride (5b, 31 mg, 0.165 mmol), and potassium carbonate (23 mg, 0.165 mmol) are refluxed in acetone (15 mL) for 3 h. After cooling down to rt, the reaction mixture is poured into brine (40 mL). The mixture is then extracted with methylene chloride (40 mL). The organic layer is dried (Na₂SO₄) and concentrated. The residue is chromatographed on a silica plate by elution with 5% methanol in methylene chloride. The desired compound is isolated as a pale yellow solid (64 mg, 97% yield).

Physical characteristics: MS (ES+) for m/z 407, 409 (M+H)⁺; ¹H NMR (CDCl₃) δ 8.55, 8.01, 7.59, 7.49, 7.31, 4.95.

### Example 3

### Preparation of 8

Compound 4 (45 mg, 0.177 mmol), 2-chloro-4′-phenylacetophenone (5c, 41 mg, 0.177 mmol), and potassium carbonate (25 mg, 0.177 mmol) are refluxed in acetone (16 mL) for 3 h. After cooling down to rt, the reaction mixture is poured into water (40 mL). The mixture is then extracted with methylene chloride (40 mL). The organic layer is dried (Na₂SO₄) and concentrated. The residue is chromatographed on a silica plate by elution with 5% methanol in methylene chloride. The desired compound is isolated as a white solid (79 mg, 99% yield).

Physical characteristics: MS (ES+) for m/z 449 (M+H)⁺; ¹H NMR (CDCl₃) δ 8.56, 8.14, 7.2-7.7, 5.05.

### Example 4

### Preparation of 9

Compound 4 (47 mg, 0.185 mmol), 2-nitro-4-(2-chloroacetyl)-acetanilide (5d, 47 mg, 0.185 mmol), and potassium carbonate (26 mg, 0.185 mmol) are refluxed in acetone (15 mL) for 3 h. After cooling down to rt, the reaction mixture is poured into water (40 mL). The mixture is then extracted with methylene chloride (40 mL). The organic layer is dried (Na₂SO₄) and concentrated. The residue is chromatographed on a silica plate by elution with 5% methanol in methylene chloride. The desired compound is isolated as a pale yellow solid (87 mg, 99% yield).

Physical characteristics: MS (ES+) for m/z 475 (M+H)⁺; ¹H NMR (CDCl₃) δ 10.63, 8.99, 8.91, 8.31, 7.58, 7.32, 4.91.

### Example 5

### Preparation of 10

Compound 4 (44 mg, 0.173 mmol), 4-(2-chloroacetyl)-acetanilide (5e, 37 mg, 0.173 mmol), and potassium carbonate (24 mg, 0.173 mmol) are refluxed in acetone (15 mL) for 3 h. After cooling down to rt, the reaction mixture is poured into water (40 mL). The mixture is then extracted with methylene chloride (40 mL). The organic layer is dried (Na₂SO₄) and concentrated. The residue is chromatographed on a silica plate by elution with 5% methanol in methylene chloride. The desired compound is isolated as a white solid (74 mg, 99% yield).

Physical characteristics: MS (ES+) for m/z 430 (M+H)⁺; ¹H NMR (CDCl₃) δ 8.57, 8.02, 7.79, 7.68, 7.61, 7.35, 4.96.

### Example 6

### Preparation of 11

Compound 4 (46 mg, 0.18 mmol), 2-chloro-4′-fluoroacetophenone (5f, 31 mg, 0.18 mmol), and potassium carbonate (25 mg, 0.18 mmol) are refluxed in acetone (15 mL) for 3 h. After cooling down to rt, the reaction mixture is poured into water (40 mL). The mixture is then extracted with methylene chloride (40 mL). The organic layer is dried (Na₂SO₄) and concentrated. The residue is chromatographed on a silica plate by elution with 5% methanol in methylene chloride. The desired compound is isolated as a white solid (70 mg, 99% yield).

Physical characteristics: MS (ES+) for m/z 391 (M+H)⁺; ¹H NMR (CDCl₃) δ 8.56, 8.11, 7.59, 7.33, 7.18, 4.97.

### Example 7

### Preparation of 12

Compound 4 (46 mg, 0.18 mmol), 2-chloroacetophenone (5g, 28 mg, 0.18 mmol), and potassium carbonate (25 mg, 0.18 mmol) are refluxed in acetone (15 mL) for 3 h. After cooling down to rt, the reaction mixture is poured into water (40 mL). The mixture is then extracted with methylene chloride (40 mL). The organic layer is dried (Na₂SO₄) and concentrated. The residue is chromatographed on a silica plate by elution with 5% methanol in methylene chloride. The desired compound is isolated as a white solid (65 mg, 99% yield).

Physical characteristics: MS (ES+) for m/z 373 (M+H)⁺; ¹H NMR (CDCl₃) δ 8.56, 8.06, 7.5-7.8, 7.33, 5.02.

### Example 8

### Preparation of 13

Compound 4 (51 mg, 0.2 mmol), 2,2′,4′-trichloroacetophenone (5 h, 45 mg, 0.2 mmol), and potassium carbonate (28 mg, 0.2 mmol) are refluxed in acetone (15 mL) for 3 h. After cooling down to rt, the reaction mixture is poured into water (40 mL). The mixture is then extracted with methylene chloride (40 mL). The organic layer is dried (Na₂SO₄) and concentrated. The residue is chromatographed on a silica plate by elution with 5% methanol in methylene chloride. The desired compound is isolated as a white solid (88 mg, 99% yield).

Physical characteristics: MS (ES+) for m/z 441, 443 (M+H)⁺; ¹H NMR (CDCl₃) δ 8.56, 7.75, 7.60, 7.48, 7.39, 7.31, 4.77.

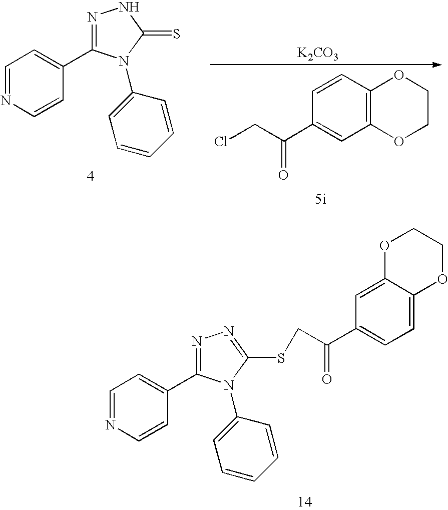

### Example 9

### Preparation of 14

Compound 4 (51 mg, 0.2 mmol), 6-chloroacetyl-1,4-benzodioxane (5i, 43 mg, 0.2 mmol), and potassium carbonate (28 mg, 0.2 mmol) are refluxed in acetone (15 mL) for 3 h. After cooling down to rt, the reaction mixture is poured into water (40 mL). The mixture is then extracted with methylene chloride (40 mL). The organic layer is dried (Na₂SO₄) and concentrated. The residue is chromatographed on a silica plate by elution with 5% methanol in methylene chloride. The desired compound is isolated as a white solid (86 mg, 99% yield).

Physical characteristics: MS (ES+) for m/z 431 (M+H)⁺; ¹H NMR (CDCl₃) δ 8.56, 7.59, 7.33, 6.95, 4.94, 4.34, 4.30.

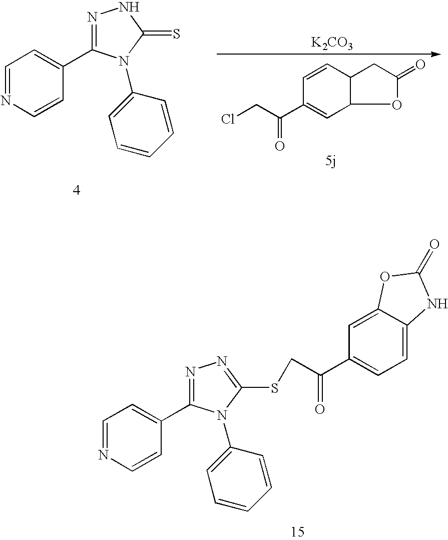

### Example 10

### Preparation of 15

Compound 4 (25 mg, 0.1 mmol), 6-chloroacetyl-2-benzoxazolinone (5j, 21 mg, 0.1 mmol), and potassium carbonate (14 mg, 0.2 mmol) are refluxed in acetone (15 mL) for 3 h. After cooling down to rt, the reaction mixture is poured into water (40 mL). The mixture is then extracted with methylene chloride (40 mL). The organic layer is dried (Na₂SO₄) and concentrated. The residue is chromatographed on a silica plate by elution with 5% methanol in methylene chloride. The desired compound is isolated as a white solid (18 mg, 42% yield).

Physical characteristics: MS (ES+) for m/z 430 (M+H)⁺; ¹H NMR (DMSO) δ 12.18, 7.93, 7.61, 7.51, 7.28, 7.26, 4.96.

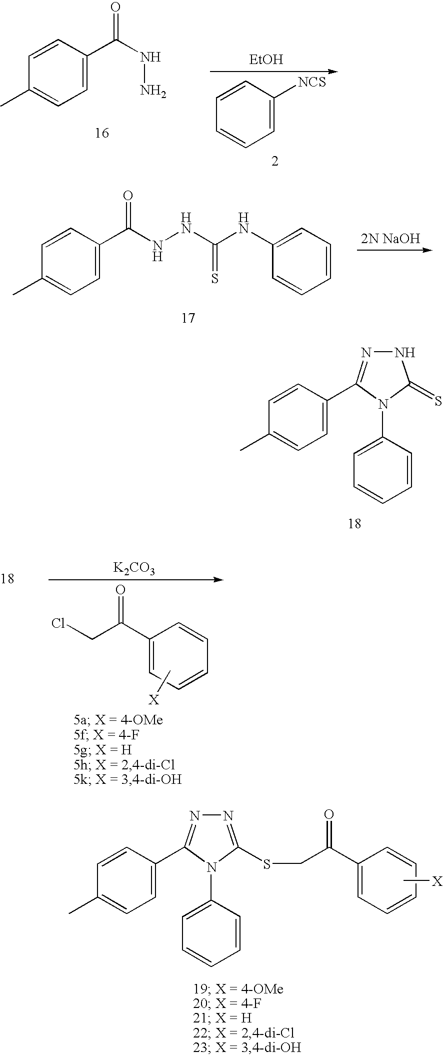

### Preparation of 17 from 16

4-Methylbenzhydrazide (16, 2.0 g, 13.3 mmol) and phenyl isothiocyanate (2, 1.6 mL, 13.3 mmol) are refluxed in ethanol for 2 h. After the mixture cooled to room temperature (rt), the white precipitate (3.62 g) is collected by filtration.

Physical characteristics: MS (ES+) for m/z 286 (M+H).

### Preparation of 18 from 17

Compound 17 (3.5 g, 12.3 mmol) is refluxed in 2 N KOH (aqueous solution, 25 mL) for 1 h. After the reaction mixture is cooled down to rt, 3 N hydrochloric acid (70 nL) is added to acidify the solution. The white precipitate is collected by filtration, washed with distilled water and dried (1.2 g).

Physical characteristics: MS (ES−) for m/z 266 (M−H).

### Example 11

### Preparation of 19

Compound 18 (47 mg, 0.176 mmol), 2-chloro-1-(4′-methoxyphenyl)-ethanone (5a, 32 mg, 0.176 mmol), and potassium carbonate (24 mg, 0.176 mmol) are refluxed in acetone (15 mL) for 2 h. After cooling down to rt, the reaction mixture is poured into brine (40 mL). The mixture is then extracted with methylene chloride (40 mL). The organic layer is dried (Na₂SO₄) and concentrated. The residue is chromatographed on a silica plate by elution with 4% methanol in methylene chloride. The desired compound is isolated as a white-solid (73 mg, 99% yield).

Physical characteristics: MS (ES+) for m/z 416 (M+H)⁺; ¹H NMR (CDCl₃) δ 8.05, 7.50, 7.29, 7.09, 6.97, 4.96, 3.89, 2.32.

### Example 12

### Preparation of 20

Compound 18 (45 mg, 0.168 mmol), 2-chloro-4′-fluoroacetophenone (5f, 29 mg, 0.168 mmol), and potassium carbonate (23 mg, 0.168 mmol) are refluxed in acetone (15 mL) for 2.5 h. After cooling down to rt, the reaction mixture is poured into water (40 mL). The mixture is then extracted with methylene chloride (40 mL). The organic layer is dried (Na₂SO₄) and concentrated. The residue is chromatographed on a silica plate by elution with 2.5% methanol in methylene chloride. The desired compound is isolated as a white solid (59 mg, 87% yield).

Physical characteristics: MS (ES+) for m/z 404 (M+H)⁺; ¹H NMR (CDCl₃) δ 8.11, 7.52, 7.29, 7.19, 7.09, 4.95, 2.33.

### Example 13

### Preparation of 21

Compound 18 (54 mg, 0.2 mmol), 2-chloroacetophenone (5g, 31 mg, 0.2 mmol), and potassium carbonate (28 mg, 0.2 mmol) are refluxed in acetone (15 mL) for 1 h. After cooling down to rt, the reaction mixture is poured into water (40 mL). The mixture is then extracted with methylene chloride (40 mL). The organic layer is dried (Na₂SO₄) and concentrated. The residue is chromatographed on a silica plate by elution with 2.5% methanol in methylene chloride. The desired compound is isolated as a white solid (62 mg, 80% yield).

Physical characteristics: MS (ES+) for m/z 386 (M+H)⁺; ¹H NMR (CDCl₃) δ 8.08, 7.62, 7.53, 7.30, 7.09, 5.01, 2.33.

### Example 14

### Preparation of 22

Compound 18 (47 mg, 0.176 mmol), 2,2′,4′-trichloroacetophenone (5 h, 39 mg, 0.176 mmol), and potassium carbonate (24 mg, 0.176 mmol) are refluxed in acetone (15 mL) for 2 h. After cooling down to rt, the reaction mixture is poured into water (40 mL). The mixture is then extracted with methylene chloride (40 mL). The organic layer is dried (Na₂SO₄) and concentrated. The residue is chromatographed on a silica plate by elution with 3.8% methanol in methylene chloride. The desired compound is isolated as a white solid (78 mg, 98% yield).

Physical characteristics: MS (ES+) for m/z 454, 456 (M+H)⁺; ¹H NMR (CDCl₃) δ 7.77, 7.2-7.6, 7.08, 4.72, 2.32.

### Example 15

### Preparation of 23

Compound 18 (43 mg, 0.16 mmol), 2-chloro-3′,4′-hydroxyacetophenone (5 k, 30 mg, 0.176 mmol), and potassium carbonate (22 mg, 0.16 mmol) are refluxed in acetone (15 mL) for 1 h. After cooling down to rt, the reaction mixture is washed with water (4×20 mL), methylene chloride (20 mL) and acetone (15 mL) successively. The white solid is dried (62 mg, 93% yield).

Physical characteristics: MS (ES−) for m/z 416 (M−H)⁺; ¹H NMR (CDCl₃) δ 10.00, 9.45, 7.56, 7.40, 7.23, 7.15, 4.82, 2.27.

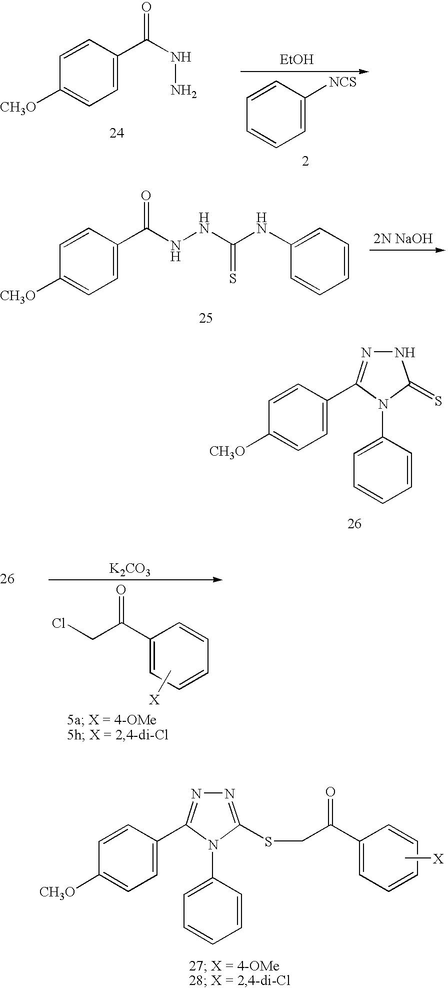

Preparation of 25 from 24

4-Methoxybenzhydrazide (24, 166 mg, 1 mmol) and phenyl isothiocyanate (2, 135 mg, 1 mmol) are refluxed in ethanol for 0.5 h. After the mixture is cooled down to rt, the white precipitate (250 mg) is collected by filtration. Physical characteristics: MS (ES+) for m/z 302 (M+H).

### Preparation of 26 from 25

Compound 25 (250 mg, 0.83 mmol) is suspended in 2 N KOH (aqueous solution, 4 mL) and the mixture is heated to 60° C. for 0.5 h. After the reaction mixture is cooled down to rt, 3 N hydrochloric acid (3 mL) is added to acidify the solution. The white precipitate is collected by filtration, washed with distilled water and dried (150 mg).

Physical characteristics: MS (ES−) for m/z 282 (M−H).

### Example 16

### Preparation of 27

Compound 26 (40 mg, 0.14 mmol), 2-chloro-1-(4′-methoxyphenyl)-ethanone (5a, 25 mg, 0.14 mmol), and potassium carbonate (24 mg, 0.176 mmol) are refluxed in acetone (8 mL) for 0.5 h. After cooling down to rt, the reaction mixture is poured into brine (10 mL). The mixture is then extracted with methylene chloride (15 mL). The organic layer is dried (Na₂SO₄) and concentrated. The residue is recrystallized from methylene chloride/diethyl ether (37 mg, white solid).

Physical characteristics: MS (ES+) for m/z 432 (M+H)⁺; ¹H NMR (CDCl₃) δ 8.03, 7.48, 7.33, 7.25, 6.96, 6.79, 4.94, 3.89, 3.78.

### Example 17

### Preparation of 28

Compound 26 (28 mg, 0.1 mmol), 2,2′,4′-trichloroacetophenone (5 h, 22 mg, 0.1 mmol), and potassium carbonate (24 mg, 0.176 mmol) are refluxed in acetone (5 mL) for 0.5 h. After cooling down to rt, the reaction mixture is poured into water (10 mL). The mixture is then extracted with methylene chloride (10 mL). The organic layer is dried (Na₂SO₄) and concentrated. The residue is chromatographed on a silica plate by elution with 5% methanol in methylene chloride. The desired compound is isolated as a white solid (37 mg, 79% yield).

Physical characteristics: MS (ES+) for m/z 470, 472 (M+H)⁺; ¹H NMR (CDCl₃) δ 7.75, 7.2-7.6, 6.78, 4.69, 3.78.

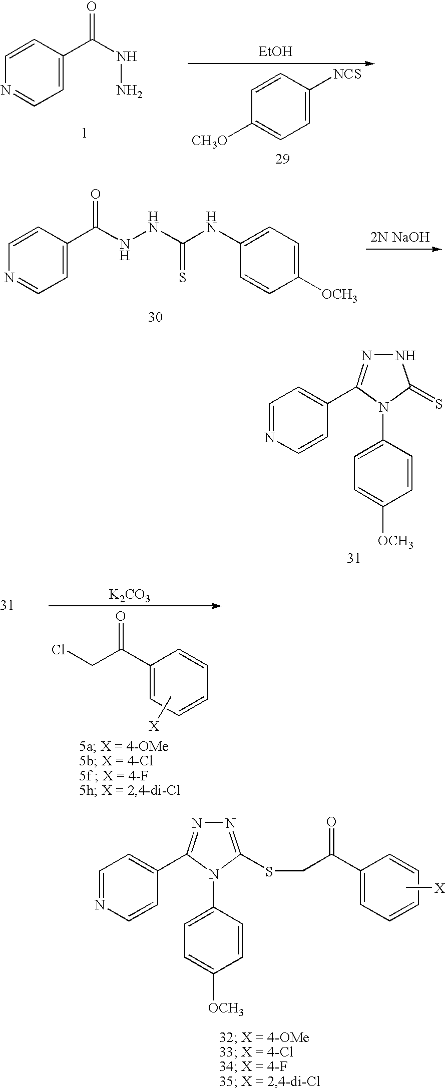

### Preparation of 30 from 1

Isonicotinic hydrazide (1, 137 mg, 1 mmol) and 4-methoxyphenyl isothiocyanate (29, 149 mg, 1 mmol) are refluxed in ethanol (6 mL) for 0.5 h. After the mixture is cooled down to rt, the white precipitate (230 mg) is collected by filtration. Physical characteristics: MS (ES+) for m/z 302 (M+H).

### Preparation of 31 from 30

Compound 30 (210 mg, 0.7 mmol) is suspended in 2 N KOH (aqueous solution, 4 mL) and the mixture is heated to 60° C. for 0.5 h. After the reaction mixture is cooled down to rt, 3 N hydrochloric acid (3 mL) is added to acidify the solution. The white precipitate is collected by filtration, washed with distilled water and dried (150 mg).

Physical characteristics: MS (ES−) for m/z 283 (M−H).

### Example 18

### Preparation of 32

Compound 31 (40 mg, 0.14 mmol), 2-chloro-1-(4′-methoxyphenyl)-ethanone (5a, 25 mg, 0.14 mmol), and potassium carbonate (24 mg, 0.176 mmol) are refluxed in acetone (8 mL) for 0.5 h. After cooling down to rt, the reaction mixture is poured into brine (10 mL). The mixture is then extracted with methylene chloride (10 mL). The organic layer is dried (Na₂SO₄) and concentrated. The residue is chromatographed on a silica plate by elution with 5% methanol in methylene chloride. The desired compound is isolated as a white solid (40 mg, 67% yield).

Physical characteristics: MS (ES+) for m/z 433 (M+H)⁺; ¹H NMR (CDCl₃) δ 8.56, 8.04, 7.34, 7.22, 7.04, 6.97, 4.96, 3.90, 3.89.

### Example 19

### Preparation of 33

Compound 31 (50 mg, 0.176 mmol), 4-chlorophenacyl chloride (5b, 40 mg, 0.211 mmol), and potassium carbonate (30 mg, 0.22 mmol) are refluxed in acetone (8 mL) for 0.5 h. After cooling down to rt, the reaction mixture is poured into brine (10 mL). The mixture is then extracted with methylene chloride (10 mL). The organic layer is dried (Na₂SO₄) and concentrated. The residue is recrystallized from methylene chloride/diethyl ether (37 mg, pale pink solid).

Physical characteristics: MS (ES+) for m/z 437, 439 (M+H)⁺; ¹H NMR (CDCl₃) δ 8.56, 8.00, 7.49, 7.34, 7.22, 7.04, 4.93, 3.90.

### Example 20

### Preparation of 34

Compound 31 (50 mg, 0.176 mmol), 2-chloro-4′-fluoroacetophenone (5f, 29 mg, 0.203 mmol), and potassium carbonate (30 mg, 0.22 mmol) are refluxed in acetone (8 mL) for 0.5 h. After cooling down to rt, the reaction mixture is poured into brine (10 mL). The mixture is then extracted with ethyl acetate (10 mL). The organic layer is dried (Na₂SO₄) and concentrated. The residue is chromatographed on a silica plate by elution with 5% methanol in methylene chloride. The desired compound is isolated as a white solid (52 mg, 70% yield).

Physical characteristics: MS (ES+) for m/z 421 (M+H)⁺; ¹H NMR (CDCl₃) δ 8.56, 8.10, 7.34, 7.1-7.3, 7.03, 4.95, 3.90.

### Example 21

### Preparation of 35

Compound 31 (40 mg, 0.14 mmol), 2,2′,4′-trichloroacetophenone (5 h, 31 mg, 0.14 mmol), and potassium carbonate (24 mg, 0.176 mmol) are refluxed in acetone (8 mL) for 0.5 h. After cooling down to rt, the reaction mixture is poured into brine (10 mL). The mixture is then extracted with ethyl acetate (10 mL). The organic layer is dried (Na₂SO₄) and concentrated. The residue is chromatographed on a silica plate by elution with 5% methanol in methylene chloride. The desired compound is isolated as a white solid (45 mg, 68% yield).

Physical characteristics: MS (ES+) for m/z 471, 473 (M+H)⁺; ¹H NMR (CDCl₃) δ 8.55, 7.74, 7.47, 7.38, 7.32, 7.20, 7.03, 4.74, 3.91.

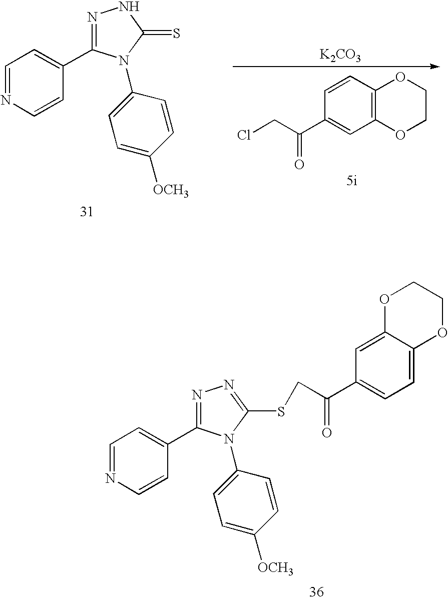

### Example 22

### Preparation of 36

Compound 31 (50 mg, 0.176 mmol), 6-chloroacetyl-1,4-benzodioxane (5i, 43 mg, 0.2 mmol), and potassium carbonate (30 mg, 0.22 mmol) are refluxed in acetone (8 mL) for 0.5 h. After cooling down to rt, the reaction mixture is poured into brine (10 mL). The mixture is then extracted with methylene chloride (10 mL). The organic layer is dried (Na₂SO₄) and concentrated. The residue is recrystallized from methylene chloride/diethyl ether (35 mg, pale pink solid).

Physical characteristics: MS (ES+) for m/z 461 (M+H)⁺; ¹H NMR (CDCl₃) δ 8.55, 7.58, 7.34, 7.22, 7.04, 6.94, 4.92, 4.34, 4.29, 3.90.

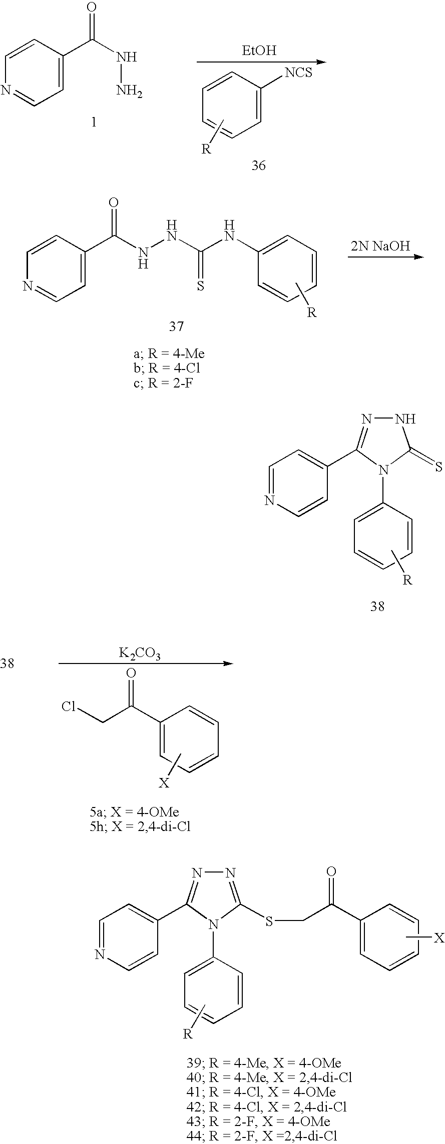

### Preparation of 37a from 1

Isonicotinic hydrazide (1, 274 mg, 2 mmol) and 4-methylphenyl isothiocyanate (36a, 300 mg, 2 mmol) are refluxed in ethanol (10 mL) for 0.5 h. After the mixture is cooled down to rt, the white precipitate (500 mg) is collected by filtration. Physical characteristics: MS (ES+) for m/z 287 (M+H).

### Preparation of 37b from 1

Isonicotinic hydrazide (1, 274 mg, 2 mmol) and 4-chlorophenyl isothiocyanate (36b, 340 mg, 2 mmol) are refluxed in ethanol (10 mL) for 0.5 h. After the mixture is cooled down to rt, the white precipitate (500 mg) is collected by filtration. Physical characteristics: MS (ES+) for m/z 307 (M+H).

### Preparation of 37c from 1

Isonicotinic hydrazide (1, 274 mg, 2 mmol) and 2-fluorophenyl isothiocyanate (36c, 306 mg, 2 mmol) are refluxed in ethanol (10 mL) for 0.5 h. After the mixture is cooled down to rt, the white precipitate (550 mg) is collected by filtration. Physical characteristics: MS (ES+) for m/z 291 (M+H).

### Preparation of 38a from 37a

Compound 37a (500 mg, 1.75 mmol) is suspended in 2 N KOH (aqueous solution, 8 mL) and the mixture is heated to 60° C. for 0.5 h. After the reaction mixture is cooled down to rt, 3 N hydrochloric acid (7 mL) is added to acidify the solution. The white precipitate is collected by filtration, washed with distilled water and dried (300 mg).

Physical characteristics: MS (ES−) for m/z 267 (M−H).

### Preparation of 38b from 37b

Compound 37b (480 mg, 1.56 mmol) is suspended in 2 N KOH (aqueous solution, 6 mL) and the mixture is heated to 60° C. for 1.5 h. After the reaction mixture is cooled down to rt, 3 N hydrochloric acid (5 mL) is. added to acidify the solution. The pale yellow precipitate is collected by filtration, washed with distilled water and dried (250 mg).

Physical characteristics: MS (ES−) for m/z 287, 289 (M−H).

### Preparation of 38c from 37c

Compound 37c (530 mg, 1.83 mmol) is suspended in 2 N KOH (aqueous solution, 10 mL) and the mixture is heated to 60° C. for 3 h. After the reaction mixture is cooled down to rt, 3 N hydrochloric acid (8 mL) is added to acidify the solution. The white precipitate is collected by filtration, washed with distilled water and dried (300 mg).

Physical characteristics: MS (ES−) for m/z 271 (M−H).

### Example 23

### Preparation of 39

Compound 38a (48 mg, 0.18 mmol), 2-chloro-1-(4′-methoxyphenyl)-ethanone (5a, 32 mg, 0.18 mmol), and potassium carbonate (30 mg, 0.22 mmol) are refluxed in acetone (8 mL) for 0.5 h. After cooling down to rt, the reaction mixture is poured into brine (10 mL). The mixture is then extracted with methylene chloride (10 mL). The organic layer is dried (Na₂SO₄) and concentrated. The residue is recrystallized from methylene chloride/diethyl ether (40 mg, pale yellow solid).

Physical characteristics: MS (ES+) for m/z 439 (M+Na)⁺; ¹H NMR (CDCl₃) δ 8.55, 8.03, 7.33, 7.17, 6.97, 4.96, 3.89, 2.48.

### Example 24

### Preparation of 40

Compound 38a (41 mg, 0.15 mmol), 2,2′,4′-trichloroacetophenone (5 h, 35 mg, 0.16 mmol), and potassium carbonate (30 mg, 0.22 mmol) are refluxed in acetone (8 mL) for 0.5 h. After cooling down to rt, the reaction mixture is poured into brine (10 mL). The mixture is then extracted with methylene chloride (10 mL). The organic layer is dried (Na₂SO₄) and concentrated. The residue is recrystallized from methylene chloride/diethyl ether (30 mg, white solid).

Physical characteristics: MS (ES+) for m/z 477 (M+Na)⁺; ¹H NMR (CDCl₃) δ 8.55, 7.73, 7.47, 7.38, 7.34, 7.31, 7.16, 4.74, 2.48.

### Example 25

### Preparation of 41

Compound 38b (50 mg, 0.17 mmol), 2-chloro-1-(4′-methoxyphenyl)-ethanone (5a, 36 mg, 0.2 mmol), and potassium carbonate (30 mg, 0.22 mmol) are refluxed in acetone (8 mL) for 0.5 h. After cooling down to rt, the reaction mixture is poured into brine (10 mL). The mixture is then extracted with methylene chloride (10 mL). The residue is chromatographed on a silica plate by elution with 5% methanol in methylene chloride. The desired compound is isolated as a white solid (43 mg, 55% yield).

Physical characteristics: MS (ES+) for m/z 437 (M+H)⁺; ¹H NMR (CDCl₃) δ 8.58, 8.02, 7.54, 7.31, 7.27, 6.97, 4.97, 3.89.

### Example 26

### Preparation of 42

Compound 38b (50 mg, 0.17 mmol), 2,2′,4′-trichloroacetophenone (5 h, 44 mg, 0.2 mmol), and potassium carbonate (30 mg, 0.22 mmol) are refluxed in acetone (8 mL) for 0.5 h. After cooling down to rt, the reaction mixture is poured into brine (10 mL). The mixture is then extracted with methylene chloride (10 mL). The organic layer is dried (Na₂SO₄) and concentrated. The residue is recrystallized from methylene chloride/diethyl ether (37 mg, white solid).

Physical characteristics: MS (ES+) for m/z 475, 477, 479 (M+H)⁺; ¹H NMR (CDCl₃) δ 8.58, 7.73, 7.55, 7.47, 7.38, 7.28, 4.76.

### Example 27

### Preparation of 43

Compound 38c (50 mg, 0.18 mmol), 2-chloro-1-(4′-methoxyphenyl)-ethanone (5a, 36 mg, 0.2 mmol), and potassium carbonate (30 mg, 0.22 mmol) are refluxed in acetone (8 mL) for 0.5 h. After cooling down to rt, the reaction mixture is poured into brine (10 mL). The mixture is then extracted with methylene chloride (10 mL). The organic layer is dried (Na₂SO₄) and concentrated. The residue is recrystallized from methylene chloride/diethyl ether (30 mg, pale yellow solid).

Physical characteristics: MS (ES+) for m/z 421 (M+H)⁺; ¹H NMR (CDCl₃) δ 8.57, 8.03, 7.60, 7.2-7.5, 6.97, 5.07, 4.92, 3.89.

### Example 28

### Preparation of 44

Compound 38c (50 mg, 0.183 mmol), 2,2′,4′-trichloroacetophenone (5 h, 44 mg, 0.2 mmol), and potassium carbonate (30 mg, 0.22 mmol) are refluxed in acetone (8 mL) for 0.5 h. After cooling down to rt, the reaction mixture is poured into brine (10 mL). The mixture is then extracted with methylene chloride (10 mL). The organic layer is dried (Na₂SO₄) and concentrated. The residue is chromatographed on a silica plate by elution with 5% methanol in methylene chloride. The desired compound is isolated as a white solid (55 mg, 65% yield).

Physical characteristics: MS (ES+) for m/z 459, 461 (M+H)⁺; ¹H NMR (DMSO) δ 8.61, 7.91, 7.79, 7.71, 7.64, 7.56, 7.50, 7.32, 4.87.

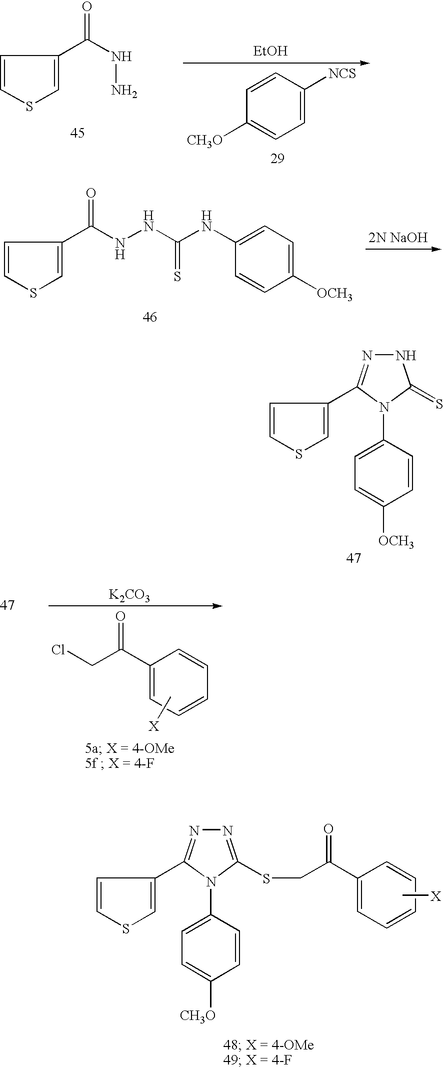

### Preparation of 46 from 45

3-Thiophenecarboxylic acid hydrazide (45, 284 mg, 2 mmol) and 4-methoxyphenyl isothiocyanate (29, 330 mg, 2 mmol) are refluxed in ethanol (10 mL) for 0.5 h. After the mixture is cooled down to rt, the white precipitate (600 mg) is collected by filtration. Physical characteristics: MS (ES+) for m/z 308 (M+H).

### Preparation of 47 from 46

Compound 46 (600 mg, 1.95 mmol) is suspended in 2 N KOH (aqueous solution, 4 mL) and the mixture is heated to 60° C. for 0.5 h. After the reaction mixture is cooled down to rt, 3 N hydrochloric acid (4 mL) is added to acidify the solution. The white precipitate is collected by filtration, washed with distilled water and dried (420 mg).

Physical characteristics: MS (ES−) for m/z 288 (M−H).

### Example 29

### Preparation of 48

Compound 47 (50 mg, 0.173 mmol), 2-chloro-1-(4′-methoxyphenyl)-ethanone (5a, 36 mg, 0.2 mmol), and potassium carbonate (30 mg, 0.22 mmol) are refluxed in acetone (8 mL) for 0.5 h. After cooling down to rt, the reaction mixture is poured into brine (10 mL). The mixture is then extracted with methylene chloride (10 mL). The organic layer is dried (Na₂SO₄) and concentrated. The residue is recrystallized from methylene chloride/diethyl ether (52 mg, white solid).

Physical characteristics: MS (ES+) for m/z 438 (M+H)⁺; ¹H NMR (CDCl₃) δ 8.61, 7.34, 7.25, 7.09, 7.05, 6.95, 4.92, 3.90, 3.88.

### Example 30

### Preparation of 49

Compound 47 (50 mg, 0.183 mmol), 4-fluoroacetophenone (5f, 34 mg, 0.2 mmol), and potassium carbonate (30 mg, 0.22 mmol) are refluxed in acetone (8 mL) for 0.5 h. After cooling down to rt, the reaction mixture is poured into brine (10 mL). The mixture is then extracted with methylene chloride (10 mL). The organic layer is dried (Na₂SO₄) and concentrated. The residue is recrystallized from methylene chloride/diethyl ether (30 mg, white solid).

Physical characteristics: MS (ES+) for m/z 426 (M+H)⁺; ¹H NMR (CDCl₃) δ 8.10, 7.0-7.4, 4.91, 3.90.

### Example 31

### Brief Bioligical Assay Description and Biological Activity of Selected Compounds

As previously described (Larsen op.cit.), CHO cells were transfected with the DAR-2 DNA on 10 cm plates (5 mg/plate) and were trypsinized 24 h after transfections and were plated at a density of 2×104 cells/well in black-walled, 96-well plates and incubated for an additional 24 hrs at 37° C./5% CO₂ either with or without pertussis toxin (PTX, 100 ng/ml). On the day of treatment, the media were aspirated, the cells were washed with HBBS/HEPES (Hank's Balanced Salt Solution, supplemented with 10 mM HEPES) and incubated with 4 μM Fluo-3AM in HBSS/HEPES, additionally supplemented with 2.5 mM probenecid to inhibit multi drug resistant pump (0.1 ml/well) for 1 h at 37° C./5% CO₂. Plates were washed twice with warm HBSS/HEPES/ probenecid buffer immediately prior to activation of the calcium response and 100 μl buffer/per well was left after the last wash. A calcium response was initiated by the addition of candidate receptor agonist compounds (2× concentration in HBSS/HEPES, 100 μl/well). The DSK-R₁/SHEP cells were loaded for 1 h at 37° C./5% CO₂ with 4 μM calcium green/0.02% pluronic acid in modified Earle's balanced salt solution containing 4 mM CaCl₂ dihydrate, 0.8 mM MgSO₄.7H₂O, 20 mM NaCl, 20 mM Tris-HEPES, 120 mM N-methyl-D-glucamine/HCl, 5.3 mM KCl, 5.6 M D-glucose, and 9 mM Tris base. Fluorescence was measured on a 96-well plate-based fluorescence imaging plate reader (FLIPR) with an argon laser (Molecular Devices, Sunnyvale, Calif.). Basal fluorescence of cells was measured for 20 seconds prior to addition of candidate agonist and the basal fluorescence level was subtracted from the response signal. The calcium signal was measured for approximately 200 seconds with readings every two seconds. Calcium ionophore A23187 was used as a control for non-receptor specific calcium release.

Selected compounds were evaluated for their anti-parasitic activity in the assay as described above. Results of the evaluations are given in Table I.

TABLE 1

| Compound | IC50 (nM) |
|---|---|
| 6 | 54 |
| 7 | 77 |
| 9 | 765 |
| 10 | 297 |
| 11 | 268 |
| 12 | 124 |
| 13 | 1200 |
| 14 | 436 |
| 15 | 4500 |
| 32 | 34 |
| 33 | 0.002 |
| 34 | 898 |
| 35 | 414 |
| 36 | 103 |
| 39 | 0.00018 |
| 40 | 4800 |
| 41 | 0.0027 |
| 43 | 0.0014 |
| 44 | 0.16 |
| 49 | 14600 |

### Example 32

### Muscle Tension Assay

As previously described (Bowman, op.cit.), a 2 cm segment of Ascaris suum muscle strip was suspended in a muscle bath filled with 37° Ascaris Ringers Solution {ARS; in mM: KCl (24.5), CaCl₂ (5.9), MgCl₂ (4.9), NaCl (4), NaC₂ H₃O₂ (125) and Tris (5); pH 7.4}. The preparations were initially set at 12-15 g of tension and allowed to stabilize for approximately 15-30 minutes before drug treatment. Test compounds were added by pipet to the bath with final concentration of 30 uM reflecting the dilution in the test chamber. Raw data was collected from the BioReport™ program. Results of evaluating selected compounds are summarized in Table II.

TABLE II

| Cmpd # | Conc. | Activity |
|---|---|---|
| 6 | 30 uM | Excitatory |

1. A compound of Formula I 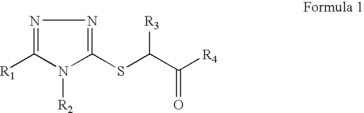
wherein:
R₁, R₂ and R₄ are independently selected from the group H, C₁-C₈ alkyl, phenyl, substituted phenyl, benzyl, substituted benzyl, heteroaryl, substituted heteroaryl, hetroarylmethylene, and substituted hetroarylmethylene;
R₃ is H, C₁-C₈ alkyl, heteroalkyl, cycloalkyl, aryl, heteroaryl;
with the proviso that the compound is not:
1-(4-methoxyphenyl)-2-[(4-phenyl-5-pyridin-4-yl-4H-1,2,4-triazol-3-yl)thio]ethanone (6);
1-(4-chlorophenyl)-2-[(4-phenyl-5-pyridin-4-yl-4H-1,2,4-triazol-3-yl)thio]ethanone (7);
1-(4-fluorophenyl)-2-[(4-phenyl-5-pyridin-4-yl-4H-1,2,4-triazol-3-yl)thio]ethanone (11);
1-phenyl-2-[(4-phenyl-5-pyridin-4-yl-4H-1,2,4-triazol-3-yl)thio]ethanone (12);
2-{[5-(4-methylphenyl)-4-phenyl-4H-1,2,4-triazol-3-yl]thio}-1-phenylethanone (21);
1-(4-methoxyphenyl)-2-{[5-(4-methoxyphenyl)-4-phenyl-4H-1,2,4-triazol-3-yl]thio}ethanone (27); or
1-(4-methoxyphenyl)-2-{[4-(4-methylphenyl)-5-pyridin-4-yl-4H-1,2,4-triazol-3-yl]thio}ethanone (39).

2. A compound of claim 1 selected from the group:
a) 1-(1,1′-biphenyl-4-yl)-2-[(4-phenyl-5-pyridin-4-yl-4H-1,2,4-triazol-3-yl)thio]ethanone;
b) N-(2-nitro-4-{[(4-phenyl-5-pyridin-4-yl-4H-1,2,4-triazol-3-yl)thio]acetyl}phenyl)acetamide;
c) N-(4-{2-[(4-phenyl-5-pyridin4-yl4H-1,2,4-triazol-3-yl)thio]acetyl}phenyl)acetamide;
d) 1-(2,4-dichlorophenyl)-2-[(4-phenyl-5-pyridin-4-yl-4H-1,2,4-triazol-3-yl)thio]ethanone;
e) 1-(2,3-dihydro-1,4-benzodioxin-6-yl)-2-[(4-phenyl-5-pyridin-4-yl4H-1,2,4-triazol-3-yl)thio]ethanone;
f) 6-{[(4-phenyl-5-pyridin-4-yl-4H-1,2,4-triazol-3-yl)thio]acetyl}-1,3-benzoxazol-2(3H)-one;
g) 1-(4-methoxyphenyl)-2-{[5-(4-methylphenyl)-4-phenyl-4H-1,2,4-triazol-3-yl]thio}ethanone;
h) 1-(4-fluorophenyl)-2-{[5-(4-methylphenyl)-4-phenyl-4H-1,2,4-triazol-3-yl]thio}ethanone;
i) 1-(2,4-dichlorophenyl)-2-{[5-(4-methylphenyl)-4-phenyl-4H-1,2,4-triazol-3-yl]thio}ethanone;
j) 1-(3,4-dihydroxyphenyl)-2-{[5-(4-methylphenyl)-4-phenyl-4H-1,2,4-triazol-3-yl]thio}ethanone;
k) 1-(2,4-dichlorophenyl)-2-{[5-(4-methoxyphenyl)-4-phenyl-4H-1,2,4-triazol-3-yl]thio}ethanone;
l) 1-(4-methoxyphenyl)-2-{[4-(4-methoxyphenyl)-5-pyridin-4-yl-4H-1,2,4-triazol-3-yl]thio}ethanone;
m) 1-(4-chlorophenyl)-2-{[4-(4-methoxyphenyl)-5-pyridin-4-yl-4H-1,2,4-triazol-3-yl]thio}ethanone;
n) 1-(4-fluorophenyl)-2-{[4-(4-methoxyphenyl)-5-pyridin-4-yl-4H-1,2,4-triazol-3-yl]thio}ethanone;
o) 1-(2,4-dichlorophenyl)-2-{[4-(4-methoxyphenyl)-5-pyridin-4-yl-4H-1,2,4-triazol-3-yl]thio}ethanone;
p) 1-(2,3-dihydro-1,4-benzodioxin-6-yl)-2-{[4-(4-methoxyphenyl)-5-pyridin-4-yl-4H-1,2,4-triazol-3-yl]thio}ethanone;
q) 1-(2,4-dichlorophenyl)-2-{[4-(4-methylphenyl)-5-pyridin-4-yl-4H-1,2,4-triazol-3-yl]thio}ethanone;
r) 2-{[4-(4-chlorophenyl)-5-pyridin-4-yl-4H-1,2,4-triazol-3-yl]thio}-1-(4-methoxyphenyl)ethanone;
s) 2-{[4-(4-chlorophenyl)-5-pyridin-4-yl-4H-1,2,4-triazol-3-yl]thio}-1-(2,4-dichlorophenyl)ethanone;
t) 2-{[4-(2-fluorophenyl)-5-pyridin-4-yl-4H-1,2,4-triazol-3-yl]thio}-1-(4-methoxyphenyl)ethanone;
u) 1-(2,4-dichlorophenyl)-2-{[4-(2-fluorophenyl)-5-pyridin-4-yl-4H-1,2,4-triazol-3-yl]thio}ethanone;
v) 1-(4-methoxyphenyl)-2-{[4-(4-methoxyphenyl)-5-thien-3-yl-4H-1,2,4-triazol-3-yl]thio}ethanone; and
w) 1-(4-fluorophenyl)-2-{[4-(4-methoxyphenyl)-5-thien-3-yl-4H-1,2,4-triazol-3-yl]thio}ethanone.

3. The method of treating parasitism by insects or helminths comprising administering to a mammal in need of such treatment a therapeutically effective amount of a compound of Formula I or a pharmaceutically acceptable salt thereof.

4. The method of claim 3 wherein the compound is:
a) 1-(4-methoxyphenyl)-2-[(4-phenyl-5-pyridin-4-yl-4H-1,2,4-triazol-3-yl)thio]ethanone;
b) 1-(4-chlorophenyl)-2-[(4-phenyl-5-pyridin-4-yl-4H-1,2,4-triazol-3-yl)thio]ethanone;
c) 1-(1,1′-biphenyl-4-yl)-2-[(4-phenyl-5-pyridin-4-yl-4H-1,2,4-triazol-3-yl)thio]ethanone;
d) N-(2-nitro-4-{[(4-phenyl-5-pyridin-4-yl-4H-1,2,4-triazol-3-yl)thio]acetyl}phenyl)acetamide;
e) N-(4-{2-[(4-phenyl-5-pyridin-4-yl-4H-1,2,4-triazol-3-yl)thio]acetyl}phenyl)acetamide;
f) 1-(4-fluorophenyl)-2-[(4-phenyl-5-pyridin-4-yl4H-1,2,4-triazol-3-yl)thio]ethanone;
g) 1-phenyl-2-[(4-phenyl-5-pyridin-4-yl-4H-1,2,4-triazol-3-yl)thio]ethanone;
h) 1-(2,4-dichlorophenyl)-2-[(4-phenyl-5-pyridin-4-yl4H-1,2,4-triazol-3-yl)thio]ethanone;
i) 1-(2,3-dihydro-1,4-benzodioxin-6-yl)-2-[(4-phenyl-5-pyridin-4-yl-4H-1,2,4-triazol-3-yl)thio]ethanone;
j) 6-{[(4-phenyl-5-pyridin-4-yl-4H-1,2,4-triazol-3-yl)thio]acetyl}-1,3-benzoxazol-2(3H)-one;
k) 1-(4-methoxyphenyl)-2-{[5-(4-methylphenyl)-4-phenyl-4H-1,2,4-triazol-3-yl]thio}ethanone;
l) 1-(4-fluorophenyl)-2-{[5-(4-methylphenyl)-4-phenyl-4H-1,2,4-triazol-3-yl]thio}ethanone;
m) 2-{[5-(4-methylphenyl)-4-phenyl-4H-1,2,4-triazol-3-yl]thio}-1-phenylethanone;
n) 1-(2,4-dichlorophenyl)-2-{[5-(4-methylphenyl)-4-phenyl-4H-1,2,4-triazol-3-yl]thio}ethanone;
o) 1-(3,4-dihydroxyphenyl)-2-{[5-(4-methylphenyl)-4-phenyl-4H-1,2,4-triazol-3-yl]thio}ethanone;
p) 1-(4-methoxyphenyl)-2-{[5-(4-methoxyphenyl)-4-phenyl-4H-1,2,4-triazol-3-yl]thio}ethanone;
q) 1-(2,4-dichlorophenyl)-2-{[5-(4-methoxyphenyl)-4-phenyl-4H-1,2,4-triazol-3-yl]thio}ethanone;
r) 1-(4-methoxyphenyl)-2-{[4-(4-methoxyphenyl)-5-pyridin-4-yl-4H-1,2,4-triazol-3-yl]thio}ethanone;
s) 1-(4-chlorophenyl)-2-{[4-(4-methoxyphenyl)-5-pyridin-4-yl-4H-1,2,4-triazol-3-yl]thio}ethanone;
t) 1-(4-fluorophenyl)-2-{[4-(4-methoxyphenyl)-5-pyridin-4-yl-4H-1,2,4-triazol-3-yl]thio}ethanone;
u) 1-(2,4-dichlorophenyl)-2-{[4-(4-methoxyphenyl)-5-pyridin-4-yl-4H-1,2,4-triazol-3-yl]thio}ethanone;
v) 1-(2,3-dihydro-1,4-benzodioxin-6-yl)-2-{[4-(4-methoxyphenyl)-5-pyridin-4-yl-4H-1,2,4-triazol-3-yl]thio}ethanone;
w) 1-(4-methoxyphenyl)-2-{[4-(4-methylphenyl)-5-pyridin-4-yl-4H-1,2,4-triazol-3-yl]thio}ethanone;
x) 1-(2,4-dichlorophenyl)-2-{[4-(4-methylphenyl)-5-pyridin-4-yl-4H-1,2,4-triazol-3-yl]thio}ethanone;
y) 2-{[4-(4-chlorophenyl)-5-pyridin-4-yl-4H-1,2,4-triazol-3-yl]thio}-1-(4-methoxyphenyl)ethanone;
z) 2-{[4-(4-chlorophenyl)-5-pyridin-4-yl-4H-1,2,4-triazol-3-yl]thio}-1-(2,4-dichlorophenyl)ethanone;
aa) 2-{[4-(2-fluorophenyl)-5-pyridin-4-yl-4H-1,2,4-triazol-3-yl]thio}-1-(4-methoxyphenyl)ethanone;
bb) 1-(2,4-dichlorophenyl)-2-{[4-(2-fluorophenyl)-5-pyridin-4-yl-4H-1,2,4-triazol-3-yl]thio}ethanone;
cc) 1-(4-methoxyphenyl)-2-{[4-(4-methoxyphenyl)-5-thien-3-yl-4H-1,2,4-triazol-3-yl]thio}ethanone; and
dd) 1-(4-fluorophenyl)-2-{[4-(4-methoxyphenyl)-5-thien-3-yl-4H-1,2,4-triazol-3-yl]thio}ethanone.

5. A composition containing a compound of Formula I 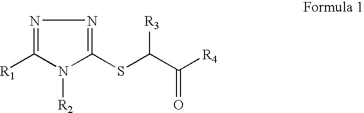
wherein:
R₁, R₂ and R₄ are independently selected from the group H, C₁-C₈ alkyl, phenyl, substituted phenyl, benzyl, substituted benzyl, heteroaryl, substituted heteroaryl, hetroarylmethylene, and substituted hetroarylmethylene;
R₃ is H, C₁-C₈ alkyl, heteroalkyl, cycloalkyl, aryl, heteroaryl.

6. A composition of claim 5 wherein the compound is:
a) 1-(4-methoxyphenyl)-2-[(4-phenyl-5-pyridin-4-yl-4H-1,2,4-triazol-3-yl)thio]ethanone;
b) 1-(4-chlorophenyl)-2-[(4-phenyl-5-pyridin-4-yl-4H-1,2,4-triazol-3-yl)thio]ethanone;
c) 1-(1,1′-biphenyl-4-yl)-2-[(4-phenyl-5-pyridin-4-yl-4H-1,2,4-triazol-3-yl)thio]ethanone;
d) N-(2-nitro-4-{[(4-phenyl-5-pyridin-4-yl4H-1,2,4-triazol-3-yl)thio]acetyl}phenyl)acetamide;
e) N-(4-{2-[(4-phenyl-5-pyridin4-yl-4H-1,2,4-triazol-3-yl)thio]acetyl}phenyl)acetamide;
f) 1-(4-fluorophenyl)-2-[(4-phenyl-5-pyridin-4-yl-4H-1,2,4-triazol-3-yl)thio]ethanone;
g) 1-phenyl-2-[(4-phenyl-5-pyridin-4-yl-4H-1,2,4-triazol-3-yl)thio]ethanone;
h) 1-(2,4-dichlorophenyl)-2-[(4-phenyl-5-pyridin-4-yl-4H-1,2,4-triazol-3-yl)thio]ethanone;
i) 1-(2,3-dihydro-1,4-benzodioxin-6-yl)2-[(4-phenyl-5-pyridin-4-yl-4H-1,2,4-triazol-3-yl)thio]ethanone;
j) 6-{[(4-phenyl-5-pyridin-4-yl-4H-1,2,4-triazol-3-yl)thio]acetyl}-1,3-benzoxazol-2(3H)-one;
k) 1-(4-methoxyphenyl)-2-{[5-(4-methylphenyl)-4-phenyl-4H-1,2,4-triazol-3-yl]thio}ethanone;
l) 1-(4-fluorophenyl)-2-{[5-(4-methylphenyl)-4-phenyl-4H-1,2,4-triazol-3-yl]thio}ethanone;
m) 2-{[5-(4-methylphenyl)-4-phenyl-4H-1,2,4-triazol-3-yl]thio}-1-phenylethanone;
n) 1-(2,4-dichlorophenyl)-2-{[5-(4-methylphenyl)-4-phenyl-4H-1,2,4-triazol-3-yl]thio}ethanone;
o) 1-(3,4-dihydroxyphenyl)-2-{[5-(4-methylphenyl)-4-phenyl-4H-1,2,4-triazol-3-yl]thio}ethanone;
p) 1-(4-methoxyphenyl)-2-{[5-(4-methoxyphenyl)-4-phenyl-4H-1,2,4-triazol-3-yl]thio}ethanone;
q) 1-(2,4-dichlorophenyl)-2-{[5-(4-methoxyphenyl)-4-phenyl-4H-1,2,4-triazol-3-yl]thio}ethanone;
r) 1-(4-methoxyphenyl)-2-{[4-(4-methoxyphenyl)-5-pyridin-4-yl-4H-1,2,4-triazol-3-yl]thio}ethanone;
s) 1-(4-chlorophenyl)-2-{[4-(4-methoxyphenyl)-5-pyridin-4-yl-4H-1,2,4-triazol-3-yl]thio}ethanone;
t) 1-(4-fluorophenyl)-2-{[4-(4-methoxyphenyl)-5-pyridin-4-yl4H-1,2,4-triazol-3-yl]thio}ethanone;
u) 1-(2,4-dichlorophenyl)-2-{[4-(4-methoxyphenyl)-5-pyridin-4-yl4H-1,2,4-triazol-3-yl]thio}ethanone;
v) 1-(2,3-dihydro-1,4-benzodioxin-6-yl)-2-{[4-(4-methoxyphenyl)-5-pyridin-4-yl-4H-1,2,4-triazol-3-yl]thio}ethanone;
w) 1-(4-methoxyphenyl)-2-{[4-(4-methylphenyl)-5-pyridin-4-yl4H-1,2,4-triazol-3-yl]thio}ethanone;
x) 1-(2,4-dichlorophenyl)-2-{[4-(4-methylphenyl)-5-pyridin-4-yl-4H-1,2,4-triazol-3-yl]thio}ethanone;
y) 2-{[4-(4-chlorophenyl)-5-pyridin-4-yl-4H-1,2,4-triazol-3-yl]thio}-1-(4-methoxyphenyl)ethanone;
z) 2-{[4-(4-chlorophenyl)-5-pyridin-4-yl-4H-1,2,4-triazol-3-yl]thio}-1-(2,4-dichlorophenyl)ethanone;
aa) 2-{[4-(2-fluorophenyl)-5-pyridin-4-yl-4H-1,2,4-triazol-3-yl]thio}-1-(4-methoxyphenyl)ethanone;
bb) 1-(2,4-dichlorophenyl)-2-{[4-(2-fluorophenyl)-5-pyridin-4-yl-4H-1,2,4-triazol-3-yl]thio}ethanone;
cc) 1-(4-methoxyphenyl)-2-{[4-(4-methoxyphenyl)-5-thien-3-yl-4H-1,2,4-triazol-3-yl]thio}ethanone; and
dd) 1-(4-fluorophenyl)-2-{[4-(4-methoxyphenyl)-5-thien-3-yl-4H-1,2,4-triazol-3-yl]thio}ethanone.
